(19) United States
(12) Patent Application Publication
(10) Pub. No.: US 2026/0278786 A1
(43) Pub. Date: Sep. 17, 2026

(54) INFORMATION PROCESSING APPARATUS, INFORMATION PROCESSING METHOD, AND NON-TRANSITORY COMPUTER READABLE MEDIUM

(71) Applicant: NEC Corporation, Tokyo (JP)

(72) Inventors: Ryoichi FURUKAWA, Tokyo (JP); Yusuke AKAMATSU, Tokyo (JP); Terumi UMEMATSU, Tokyo (JP)

(73) Assignee: NEC Corporation, Tokyo (JP)

(21) Appl. No.: 19/557,362

(22) Filed: Mar. 5, 2026

(30) Foreign Application Priority Data
Mar. 14, 2025 (JP) 2025-041809

(51) Int. Cl. G06T 7/00; A61B 5/024; G06T 7/11; G16H 50/30

(52) U.S. Cl. CPC G06T 7/0012; A61B 5/02427; G06T 7/11; G16H 50/30; G06T 2207/10016; G06T 2207/30004; G06T 2207/30201

(57) ABSTRACT

An information processing apparatus includes a pulse wave estimation unit configured to estimate a pulse wave of a living body as an electrocardiogram estimation target based on a video obtained by capturing the living body as the electrocardiogram estimation target, using a machine learning model trained using, as training data, a combination of a video obtained by capturing a living body and measurement data of a pulse wave of the living body. The information processing apparatus includes an electrocardiogram estimation unit configured to estimate an electrocardiogram of the living body as the electrocardiogram estimation target based on the estimated pulse wave. The information processing apparatus can assist in decision-making based on the electrocardiogram estimation result.

### INCORPORATION BY REFERENCE

This application is based upon and claims the benefit of priority from Japanese patent application No. 2025-041809, filed on Mar. 14, 2025, the disclosure of which is incorporated herein in its entirety by reference.

### TECHNICAL FIELD

The present disclosure relates to an information processing apparatus, an information processing method, and a non-transitory computer readable medium.

### BACKGROUND ART

An electrocardiogram may be estimated.

For example, JP 2022-169643 A describes outputting a signal equivalent to an electrocardiogram signal based on a biological vibration signal acquired by using a piezoelectric sensor.

### SUMMARY

It is preferable that an electrocardiogram can be estimated in a non-contact manner with a living body as an electrocardiogram estimation target such as a person as the electrocardiogram estimation target.

An example object of the present disclosure is to provide an information processing apparatus, an information processing method, a program that can solve the above problem.

According to a first example aspect of the present disclosure, an information processing apparatus includes pulse wave estimation means for estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body, and electrocardiogram estimation means for estimating an electrocardiogram of the living body as the electrocardiogram estimation target based on the estimated pulse wave.

According to a second example aspect of the present disclosure, an information processing method includes, by a computer, estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body, and estimating an electrocardiogram of the living body as the electrocardiogram estimation target based on the estimated pulse wave.

According to a third example aspect of the present disclosure, a program causes a computer to execute estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body, and estimating an electrocardiogram of the living body as the electrocardiogram estimation target based on the estimated pulse wave.

According to the aspects of the present disclosure, it is possible to estimate an electrocardiogram in a non-contact manner with a living body as an estimation target of the electrocardiogram.

### BRIEF DESCRIPTION OF THE DRAWINGS

FIG. 1 is a diagram illustrating an example of an overall configuration of an electrocardiogram estimation system according to the present disclosure;

FIG. 2 is a block diagram illustrating a functional configuration of an information processing apparatus according to the present disclosure;

FIG. 3 is a diagram illustrating an example of processing performed by a pulse wave estimation unit according to the present disclosure;

FIG. 4 is a diagram illustrating an example of input/output of data in an electrocardiogram estimation model according to the present disclosure;

FIG. 5 is a diagram illustrating an example of a configuration of an electrocardiogram estimation unit according to the present disclosure;

FIG. 6 is a diagram illustrating an example of processing performed by an STN used by an electrocardiogram estimation unit according to the present disclosure;

FIG. 7 is a diagram illustrating an example of processing performed by an Attention Network used by the electrocardiogram estimation unit according to the present disclosure;

FIG. 8 is a diagram illustrating an example of processing performed by an Encoder/Decoder used by the electrocardiogram estimation unit according to the present disclosure;

FIG. 9 is a diagram illustrating another example of input/output of data in the electrocardiogram estimation model according to the present disclosure;

FIG. 10 is a diagram illustrating another example of the configuration of the electrocardiogram estimation unit according to the present disclosure;

FIG. 11 is a diagram illustrating an example of training a machine learning model used by the electrocardiogram estimation unit according to the present disclosure;

FIG. 12 is a diagram illustrating an example of a configuration of an information processing apparatus according to the present disclosure;

FIG. 13 is a diagram illustrating an example of a procedure of processing in an information processing method according to the present disclosure; and

FIG. 14 is a block diagram illustrating an example of a configuration of a computer according to the present disclosure.

### EXAMPLE EMBODIMENT

Hereinafter, example embodiments will be described with reference to the drawings.

Hereinafter, a character with a circumflex may be indicated by adding “\hat{ }” next to the character. For example, p with a circumflex is also written as p\hat{ }.

### First Example Embodiment

### [Overall Configuration]

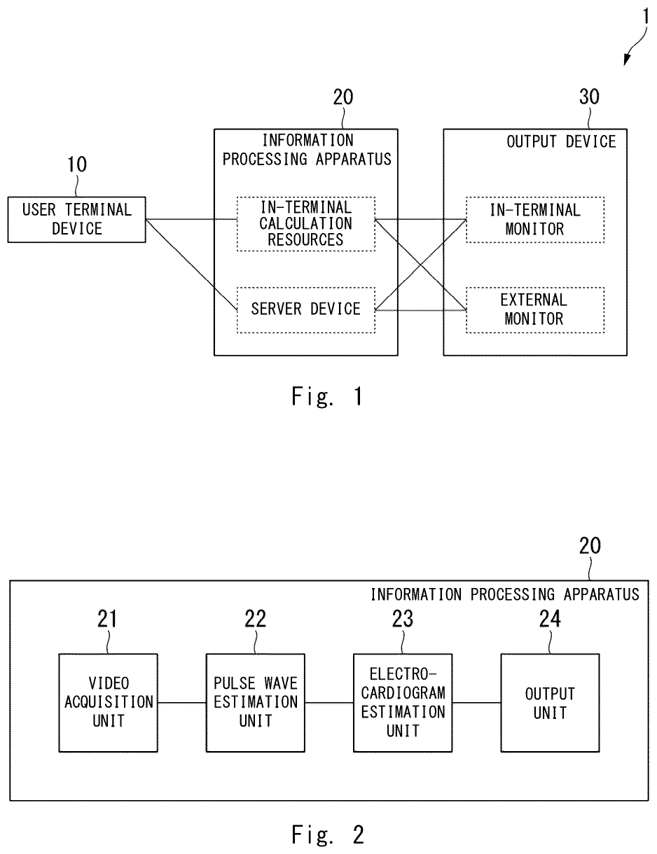

FIG. 1 is a diagram illustrating an example of an overall configuration of an electrocardiogram estimation system to which an information processing apparatus according to the present disclosure is applied.

An electrocardiogram estimation system 1 includes a user terminal device 10, an information processing apparatus 20, and an output device 30 with which a user confirms a result.

The user terminal device 10 is operated by a user (user of the electrocardiogram estimation system 1) who is an estimation target of an electrocardiogram, and is used to capture a video of the face of the user. The video here is a video. The user is an example of a living body as an electrocardiogram estimation target.

The user terminal device 10 may be configured by using, for example, a computer configured as a communication terminal such as a smartphone or a tablet terminal held by a user, or may be configured by using a device with a built-in camera. The user terminal device 10 may output a video to the information processing apparatus 20 through a communication network such as the Internet. Alternatively, calculation resources in the user terminal device 10 may be used as the information processing apparatus 20, and a camera in the user terminal device 10 may output a video to the calculation resources as the information processing apparatus 20.

The information processing apparatus 20 generates a spatiotemporal map by using RGB signals (Red Green Blue Code) extracted from a face video of the user, and estimates a pulse waveform from the generated spatiotemporal map. The pulse waveform here may be a pulse wave for one cycle. The information processing apparatus 20 estimates an electrocardiogram waveform from the estimated pulse waveform. The electrocardiogram waveform here may be an electrocardiogram for one cycle. For example, the information processing apparatus 20 may estimate the electrocardiogram waveform from information such as an amplitude and a shape of the pulse waveform.

The pulse wave is waveform data obtained by capturing a pressure change or a volume change occurring in a blood flow in a blood vessel according to the heartbeat of a heart. Photoplethysmography (PPG) is generally used as a method of measuring a pulse wave, and the pulse wave may also be referred to as PPG.

An electrocardiogram (ECG) is waveform data obtained by capturing an electrical motion signal of the heart. In the case of measuring an electrocardiogram, a voltage change due to a cardiac cycle of the heart (or heart beat) is measured. The electrocardiogram (ECG signal) includes three main components, that is, a P-wave representing atrial depolarization, a QRS complex representing ventricular depolarization, and a T-wave representing ventricular repolarization.

Since the electrocardiogram is used as a gold standard by physicians for diagnosing and preventing cardiovascular diseases, accurate and continuous measurement of an electrocardiogram signal is very important for diagnosing and preventing cardiovascular diseases. On the other hand, in the standard 12-lead electrocardiogram generally used as an electrocardiogram measurement method, a dedicated and expensive device is required, and an electrode needs to be directly attached to the skin. Therefore, the method of the standard 12-lead electrocardiogram is inconvenient for daily measurement of an electrocardiogram, and a technique capable of more easily measuring an electrocardiogram is desired.

On the other hand, the electrocardiogram estimation system 1 estimates an electrocardiogram by performing processing using a video of the user. The electrocardiogram estimation system 1 can be configured by using, for example, a combination of a camera and a personal computer (PC) and general-purpose hardware such as a smartphone or a tablet terminal. According to the electrocardiogram estimation system 1, it is possible to estimate an electrocardiogram with relatively simple work of capturing a face of a user, in a non-contact manner with the user.

The information processing apparatus 20 may calculate an index regarding a health condition of the user by using the estimated pulse waveform and/or the electrocardiogram waveform. The information processing apparatus 20 may be configured by using, for example, a server device or the like, and may communicate with the user terminal device 10 and the output device 30 by using the Internet or the like. Alternatively, in a case where a computer configured as a communication terminal, such as a smartphone or a tablet terminal, is used as the user terminal device 10, calculation resources inside the communication terminal may be used as the information processing apparatus 20. As described above, the user terminal device 10 and the information processing apparatus 20 may be integrally configured.

The information processing apparatus 20 is not limited to estimating a person's electrocardiogram; it may estimate an animal's electrocardiogram.

The output device 30 displays an electrocardiogram waveform estimated by the information processing apparatus 20. In a case where a computer configured as a communication terminal, such as a smartphone or a tablet terminal, is used as the user terminal device 10, the communication terminal may also be used as the output device 30. As described above, the user terminal device 10 and the output device 30 may be integrally configured. Alternatively, a monitor outside the user terminal device 10 may be used as the output device 30.

The output device 30 may communicate with the information processing apparatus 20 via the Internet or the like. Alternatively, in a case where the communication terminal used as the user terminal device 10 is also used as the output device 30, computational resources in the terminal device may be used as the information processing apparatus 20. As described above, the output device 30 and the information processing apparatus 20 may be integrally configured. Furthermore, the user terminal device 10, the information processing apparatus 20, and the output device 30 may be integrally configured, for example, by using one smartphone, tablet terminal, or the like as the user terminal device 10, the information processing apparatus 20, and the output device 30.

### [Functional Configuration]

FIG. 2 is a block diagram illustrating an example of a functional configuration of the information processing apparatus 20. The information processing apparatus 20 includes a video acquisition unit 21, a pulse wave estimation unit 22, an electrocardiogram estimation unit 23, and an output unit 24.

The video acquisition unit 21 acquires a video of a face captured by the user terminal device 10. The video acquisition unit 21 may have a communication function and receive a video of a face from the user terminal device 10.

Alternatively, the video acquisition unit 21 may have a function of the user terminal device 10 and capture a video of a face.

The video acquisition unit 21 outputs the acquired video of the face to the pulse wave estimation unit 22.

The pulse wave estimation unit 22 estimates a pulse waveform shape from a video of the face, which has been output from the video acquisition unit 21. The pulse wave estimation unit 22 is an example of pulse wave estimation means.

The pulse wave estimation unit 22 outputs the estimated pulse waveform shape to the electrocardiogram estimation unit 23.

The electrocardiogram estimation unit 23 estimates an electrocardiogram shape from the pulse waveform shape output from the pulse wave estimation unit 22. The electrocardiogram estimation unit 23 is an example of electrocardiogram estimation means.

The electrocardiogram estimation unit 23 outputs the estimated electrocardiogram waveform to the output unit 24.

The output unit 24 outputs the electrocardiogram waveform to the output device 30. The output unit 24 may have a communication function and transmit the electrocardiogram waveform to the output device 30.

Alternatively, the output unit 24 may have a function of the output device 30 and display the electrocardiogram waveform.

### [Processing Flow]

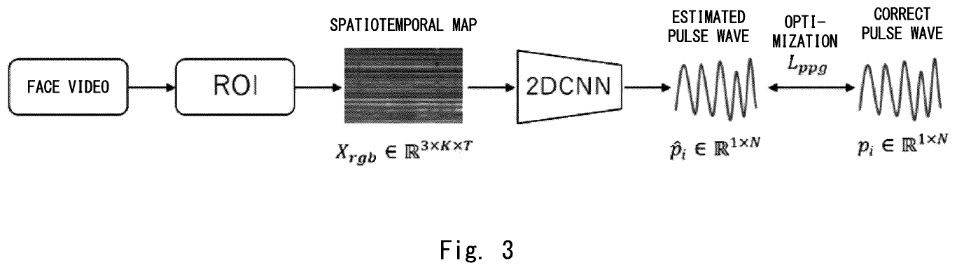

FIG. 3 is a diagram illustrating an example of processing performed by the pulse wave estimation unit 22. The pulse wave estimation unit 22 estimates the shape of a pulse wave based on the acquired video of the face.

Specifically, the pulse wave estimation unit 22 applies face detection to a face video and detects (estimates) facial feature points.

Then, the pulse wave estimation unit 22 extracts (cuts out) a region of interest (ROI) of 16 pixels×14 pixels with respect to a region under the eyes by using feature points of the eye among the detected facial feature points. However, the size of the ROI extracted by the pulse wave estimation unit 22 is not limited to a specific size. The pulse wave estimation unit 22 may extract the ROI with respect to a region other than the region under the eyes.

The pulse wave estimation unit 22 generates a spatiotemporal map X_{rgb}∈R^{3×K×T} by using an RGB time-series signal in which a plurality of extracted ROIs are arranged. Here, R^{3×K×T} represents a real space of 3×K×T dimensions. “3” of “R^{3×K×T}” indicates the number of channels of RGB, “K” indicates the number of ROIs (the number of time-series signals cut out in the ROI), and “T” indicates the number of frames of the video.

The pulse wave estimation unit 22 estimates a pulse waveform shape by using a learned deep learning model. Estimation of the pulse waveform shape performed by the pulse wave estimation unit 22 can be regarded as estimating a pulse wave based on the region cut out based on the detected feature points. The estimation of the pulse waveform shape performed by the pulse wave estimation unit 22 can also be regarded as estimating a pulse wave of a living body surface based on a color change in the living body surface indicated by the cut-out image.

During training the deep learning model used by the pulse wave estimation unit 22, data obtained by combining a face video with a pulse wave of a finger of the same person as the face video collected by using a pulse oximeter, as a correct answer, can be used as training data. Training of the machine learning model here is to adjust (optimize) a value of a parameter of the machine learning model by using the training data. Training of the machine learning model may also be referred to as learning of the machine learning model.

As the deep learning model used by the pulse wave estimation unit 22, in order to use the spatiotemporal map X_{rgb} as an input, a 2DCNN (convolutional neural network that performs two-dimensional convolution) that performs feature extraction in a two-dimensional direction by the number of ROIs and the number of frames of a video may be used. PhysNet2D may be used as an example of the 2DCNN, and is not limited to this. As a model used by the pulse wave estimation unit 22, another CNN or a Transformer may be used. For example, Y. Akamatsu, T. Umematsu, and H. Imaoka. CalibrationPhys: Self-supervised video-based heart and respiratory rate measurements by calibrating between multiple cameras. IEEE Journal of Biomedical and Health Informatics, 28:1460-1471, 2024. below is referred for PhysNet2D.

Here, as an example of a 3DCNN, a case where waveforms of a heart rate and a pulse wave are estimated from a video of a face by using a neural network called PhysNet will be considered.

In the case of using the 3DCNN such as PhysNet, input data in three-dimensional directions of the height of the face image, the width of the face image, and the number of frames of the video is used, and a calculation time is required as compared with the case of using the 2DCNN.

Therefore, in the information processing apparatus 20, a 2DCNN whose number of dimensions is smaller than three dimensions is used. As a result, the calculation time is expected to be relatively short.

In order to generate a pulse wave in which a change in the value or shape of the amplitude of the pulse wave is more accurately reflected, a loss function L_{ppg} represented by Formula (1) can be used as a loss function in training of the deep learning model used by the pulse wave estimation unit 22.

$$\begin{matrix}[ Mathematical formula 1 ] & \\ {L}_{ppg} = \frac{α}{T} {\sum }_{i = 0}^{T} {‖ {p}_{i} - {\hat{p}}_{i} ‖}^{2} + \frac{β}{T} {\sum }_{i = 0}^{T} {‖ {p}_{i}^{′} - \hat{{p}_{i}^{′}} ‖}^{2} + \frac{γ}{T} {\sum }_{i = 0}^{T} {‖ {p}_{i}^{″} - \hat{{p}_{i}^{″}} ‖}^{2} & ( 1 )\end{matrix}$$

Here, pᵢ represents a pulse wave set to be a correct answer, and p\hat{ }ᵢ represents an estimated pulse wave. p′ᵢ represents a first-order differentiated pulse wave, and p″ᵢ represents a second-order differentiated pulse wave. α, β, and γ represent hyperparameters. The information processing apparatus 20 trains the deep learning model in such a way that the value of the loss function represented by Formula (1) becomes as small as possible.

The pulse wave estimation unit 22 outputs the estimated pulse wave to the electrocardiogram estimation unit 23.

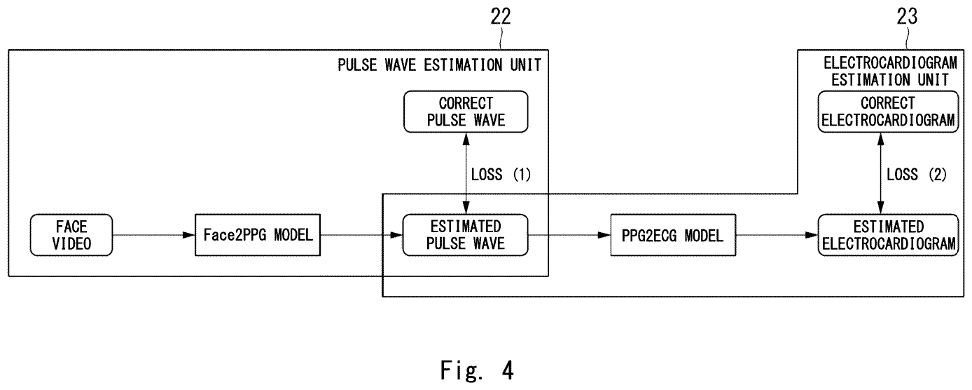

FIG. 4 is a diagram illustrating an example of input/output of data in an electrocardiogram estimation model. Here, the electrocardiogram estimation model is a model for estimating an electrocardiogram from a video of a face. In the example of FIG. 4, the electrocardiogram estimation model includes a combination of a Face2PPG model for estimating a pulse wave from a video of a face and a PPG2ECG model for estimating an electrocardiogram from the pulse wave.

The deep learning model that is used by the pulse wave estimation unit 22 and has been described above is an example of a Face2PPG model.

The PPG2ECG model is a model used by the electrocardiogram estimation unit 23 to estimate an electrocardiogram from a pulse wave. The electrocardiogram estimation unit 23 estimates the electrocardiogram based on the estimated pulse wave p\hat{ }ᵢ∈R^{1×T}. Here, R^{1×T} represents a 1×T-dimensional real space. The electrocardiogram estimation unit 23 predicts parameters of an electrocardiogram with reference to information such as a change in the amplitude and the shape of the pulse wave, and estimates the entire shape with the deep learning model.

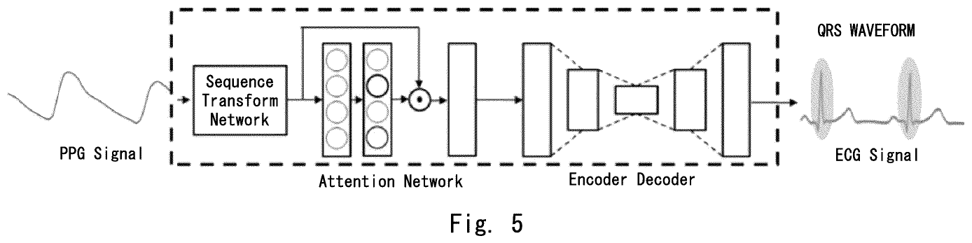

FIG. 5 is a diagram illustrating an example of a configuration of the electrocardiogram estimation unit 23.

The electrocardiogram estimation unit 23 uses a deep learning model including a Sequence Transform Network (STN), an Attention Network, and an Encoder/Decoder.

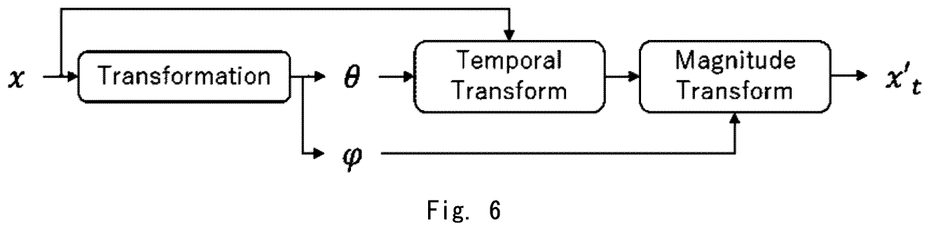

FIG. 6 is a diagram illustrating an example of processing performed by the STN used by the electrocardiogram estimation unit 23.

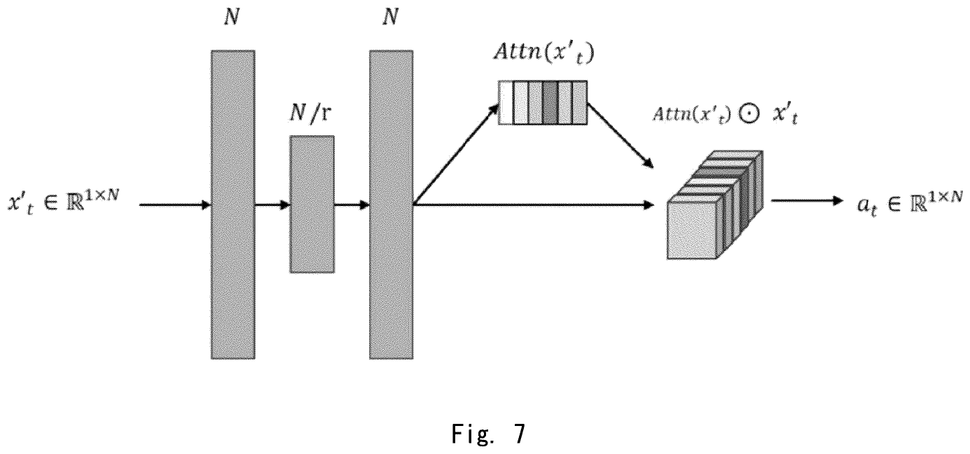

FIG. 7 is a diagram illustrating an example of processing performed by the Attention Network used by the electrocardiogram estimation unit 23.

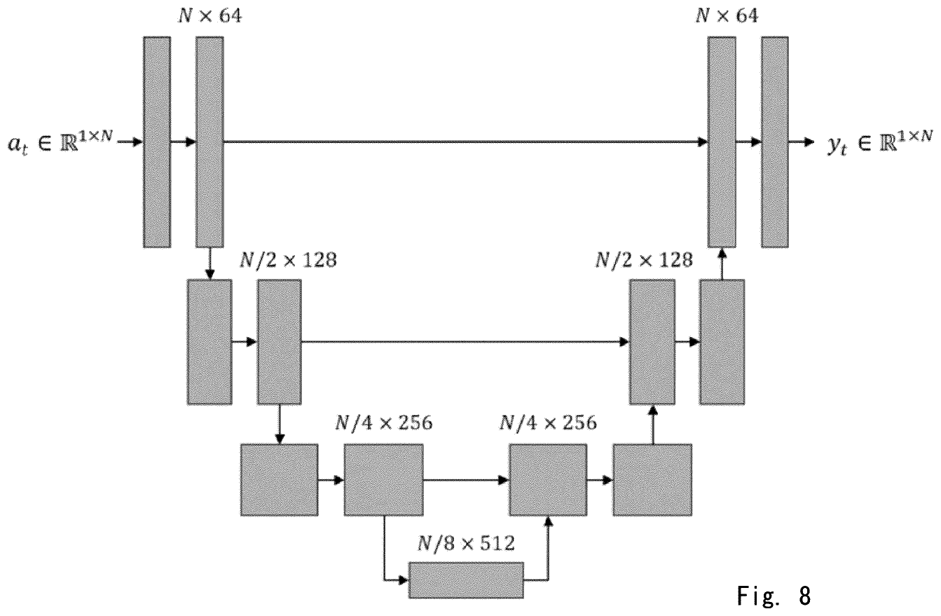

FIG. 8 is a diagram illustrating an example of processing performed by the Encoder/Decoder used by the electrocardiogram estimation unit 23.

The STN has a role of preprocessing such as conversion of the amplitude and the phase of a pulse wave estimated from an RGB signal. The Attention Network has a role of emphasizing features of the pulse wave, which are important for conversion into an electrocardiogram. The Encoder/Decoder has a role of converting an input pulse wave into an electrocardiogram.

Both the Attention Network and the Encoder/Decoder are configured as convolutional neural network (CNN). Here, CNN-based networks are sensitive to changes in input amplitude and phase. For example, depending on the communication terminal used as the user terminal device 10 and an image capturing environment, there is a high possibility that the pulse wave estimated from the RGB signal extracted from the acquired video is different in amplitude and phase, and there is a high possibility that the pulse wave adversely affects the estimation of the electrocardiogram. On the other hand, the STN used by the electrocardiogram estimation unit 23 indicates a network that adjusts the amplitude and phase of an input by using parameters θ and φ that can be learned from the pulse wave input from the pulse wave estimation unit 22. Specifically, the STN performs conversion represented by Formula (2).

$$\begin{matrix}[ Mathematical formula 2 ] & \\ \begin{matrix}{t}^{′} = θ [ \begin{matrix}t \\ 1\end{matrix} ] = [ \begin{matrix}{θ}_{1} & {θ}_{0}\end{matrix} ] [ \begin{matrix}t \\ 1\end{matrix} ] , \\ {x}_{t}^{′} = φ [ \begin{matrix}{x}_{t} \\ 1\end{matrix} ] = [ \begin{matrix}{φ}_{1} & {φ}_{0}\end{matrix} ] [ \begin{matrix}{x}_{t} \\ 1\end{matrix} ]\end{matrix} \} & ( 2 )\end{matrix}$$

Here, t represents an original time point (time), and t′ represents a converted time point. xₜ represents a pulse wave signal output from the pulse wave estimation unit 22, and x′ₜ represents a converted pulse wave signal.

The signal converted by the STN is output to the Attention Network. In the Attention Network, a portion set as being important in electrocardiogram estimation is emphasized. The Attention Network generates a weight vector having the same amplitude as an input size T by passing an input signal through two linear conversion layers and a softmax function that converts an output value into a numerical value within a range from 0 to 1. The Attention Network outputs a vector at emphasized by multiplying the weight vector by an original input for each element. Specifically, the Attention Network performs conversion represented by Formula (3).

$$\begin{matrix}[ Mathematical formula 3 ] & \\ \begin{matrix}{a}_{t} = Attn ( {x}_{t}^{′} ) ⊙ {x}_{t}^{′} , \\ Attn ( {x}_{t}^{′} ) = softmax ( {FC}_{\frac{T}{r} \to T} ( {FC}_{T \to \frac{T}{r}} ( {x}_{t}^{′} ) ) )\end{matrix} \} & ( 3 )\end{matrix}$$

Here, an operator indicated by a dot (⋅) in a circle (∘) indicates a Hadamard Product. Assuming that the number of elements of an input vector [x′ₜ] is T, the number of elements of the weight vector Attn(x′ₜ) is also T. In the Hadamard product of Formula (3), the input vector [x′ₜ] and the weight vector Attn(x′ₜ) are multiplied for each element (that is, for each index t).

r represents an integer representing a reduction rate of the dimension.

The pulse wave emphasized by the Attention Network is output to the Encoder/Decoder and converted into an electrocardiogram. Processing performed by the Encoder/Decoder is represented by Formula (4).

$$\begin{matrix}[ Mathematical formula 4 ] & \\ {y}_{t} = Decoder ( Encoder ( {a}_{t} ) ) & ( 4 )\end{matrix}$$

yₜ represents an estimated electrocardiogram.

In the example illustrated in FIG. 8, the Encoder/Decoder uses one-dimensional convolution with a kernel size of 31 for each of six layers, halves the amplitude of a signal every two layers, and doubles the number of dimensions. The Encoder/Decoder passes through a parametric rectified linear unit (PReLU) of an activation function after each convolution, and uses Batch Normalization as a normalization layer. An encoder performs Skip Connection for sending features extracted by the encoder to a decoder for each two layers. The decoder adds the features sent by Skip Connection for each element.

In the present disclosure, above-described Encoder/Decoder has been described as an example, but a model of more layers and kernel sizes may be used, or a model based on a Transformer may be used.

During training the PPG2ECG model (Training of the STN, the Attention Network, and the Encoder/Decoder), data obtained by combining the pulse wave and the electrocardiogram of the same person as the pulse wave as a correct answer can be used as training data.

In a case where the PPG2ECG model alone is trained, measurement data of the pulse wave can be used as training data. On the other hand, in a case where the entire electrocardiogram estimation model is trained, a pulse wave (pulse wave estimated from a video) output by the Face2PPG model can be regarded as being used for training data of the PPG2ECG model.

A loss function L_{ecg} represented by Formula (5) can be used to train the PPG2ECG model.

$$\begin{matrix}[ Mathematical formula 5 ] & \\ {L}_{ecg} = {\sum }_{i = 0}^{T} \vert {y}_{i} - {\hat{y}}_{i} \vert ( 1 + β {\sum }_{k = 1}^{{K}_{i}} {e}^{- \frac{{( i - {c}_{k} )}^{2}}{2 {δ}^{2}}} ) & ( 5 )\end{matrix}$$

Kᵢ indicates the number of Rpeaks of the electrocardiogram. Here, Rpeaks captured an electrical signal in which ventricular activation in the heart, and Kᵢ is the number of Rpeak events counted over a time interval T. cₖ indicates time of the k-th Rpeak.

y\hat{ }ᵢ indicates the ground-truth electrocardiogram. For example, measurement data of the electrocardiogram is used as y\hat{ }ᵢ.

e represents the Napier's constant.

δ is a hyperparameter for controlling the variance of the Gaussian distribution. “e^{(-(i-ck)2/(2δ2))}” is a partial formula for forming a Gaussian distribution centered on the Rpeak of the electrocardiogram, and the distribution is sharp if δ is small and flat if δ is large.

The information processing apparatus 20 trains the PPG2ECG model to minimize the loss function represented by Formula (5).

The electrocardiogram output by the Encoder/Decoder is output to the output unit 24. The electrocardiogram estimation unit 23 ends the processing of estimating the electrocardiogram.

### MODIFIED EXAMPLES

Next, modified examples of the information processing apparatus 20 will be described. Except for the matters described in the respective modified examples, the same as described above is applied. A plurality of modified examples may be combined and implemented.

### Modified Example 1

The information processing apparatus 20 may convert the estimated electrocardiogram into a pulse wave again by training the machine learning model, and bring the pulse wave converted again from the electrocardiogram close to the pulse wave estimated by the pulse wave estimation unit 22. As a result, it is expected that the electrocardiogram estimation unit 23 can perform more accurate electrocardiogram estimation.

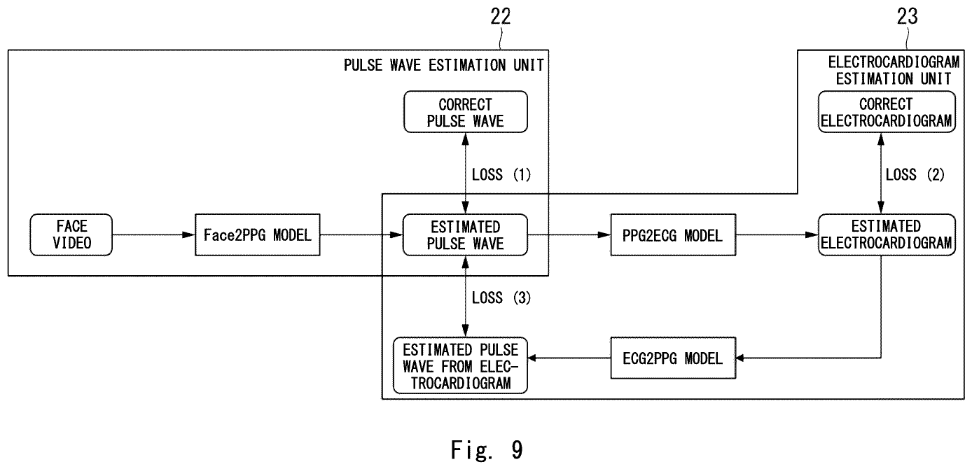

FIG. 9 is a diagram illustrating an example of input/output of data in the electrocardiogram estimation model.

In the example of FIG. 9, the electrocardiogram estimation model further includes an ECG2PPG model in addition to the machine learning model (the Face2PPG model and the PPG2ECG model) in the example of FIG. 4. In the example of FIG. 9, the PPG2ECG model and the ECG2PPG model are examples of models used by the electrocardiogram estimation unit 23 at time of training of the machine learning model.

The ECG2PPG model estimates the pulse wave from the electrocardiogram estimated by the PPG2ECG model.

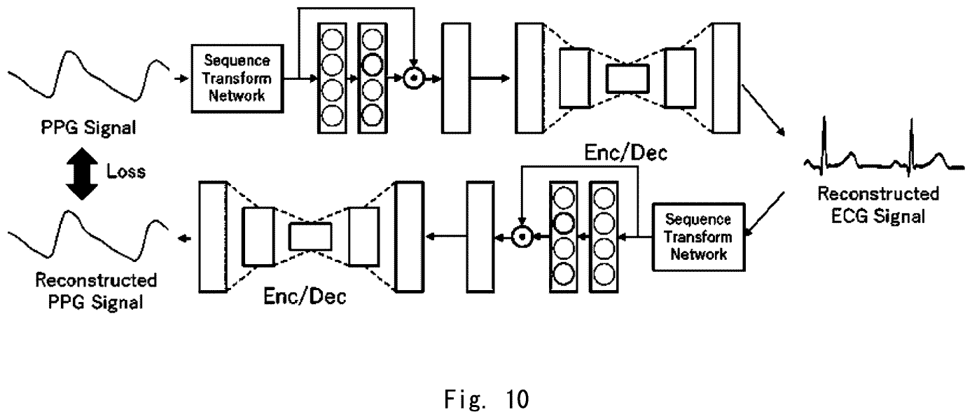

FIG. 10 is a diagram illustrating an example of the configuration of the electrocardiogram estimation unit 23.

As illustrated in FIG. 10, a pulse wave is generated from the electrocardiogram by using the STN, the Attention Network, and the Encoder/Decoder for the estimated electrocardiogram. A combination of the STN, the Attention Network, and the Encoder/Decoder is an example of the ECG2PPG model. Generating the pulse wave from the electrocardiogram here can be regarded as converting the pulse wave from the electrocardiogram again or restoring the pulse wave from the electrocardiogram.

By training the machine learning model (in particular, training PPG2ECG model) in such a way that the pulse wave estimated from the electrocardiogram is brought close to the pulse wave of the input to the electrocardiogram estimation unit 23, it is ensured that the electrocardiogram output by the PPG2ECG model can be brought back to the original pulse wave, and a relationship between the estimated electrocardiogram and the pulse wave increases. In this respect, it is expected that more accurate electrocardiogram generation can be achieved.

A loss function L_{reppg} represented by Formula (6) can be used to train the ECG2PPG model.

$$\begin{matrix}[ Mathematical formula 6 ] & \\ {L}_{reppg} = {\sum }_{i = 0}^{T} ( \vert {y}_{i} - {\hat{y}}_{i} \vert + \vert {y}_{i}^{′} - \hat{{y}_{i}^{′}} \vert + \vert {y}_{i}^{″} - \hat{{y}_{i}^{″}} \vert ) & ( 6 )\end{matrix}$$

Here, yᵢ represents the reconstructed pulse wave, and y\hat{ }ᵢ represents the pulse wave output from the pulse wave estimation unit 22.

The information processing apparatus 20 performs training of the entire electrocardiogram estimation model (training of the Face2PPG model, the PPG2ECG model, and the ECG2PPG model) by using the loss function represented by Formula (6), in such a way that the electrocardiogram estimated from the pulse wave generated from the face video has a waveform that can be brought back to the original pulse wave. In particular, the information processing apparatus 20 performs not only adjustment of parameter values of the ECG2PPG model but also adjustment of parameter values of the PPG2ECG model in such a way that the value of the loss function represented by Formula (6) becomes as small as possible.

At time of operation of the information processing apparatus 20, the electrocardiogram estimation unit 23 may not include the ECG2PPG model.

### Modified Example 2

It is expected that the electrocardiogram estimation unit 23 can perform more accurate electrocardiogram estimation by performing contrastive learning by using an electrocardiogram waveform output from the Encoder/Decoder.

In the contrastive learning here, it is determined that the electrocardiogram waveform generated by using the pulse wave estimated from the face video is similar to the electrocardiogram waveform generated by using the pulse wave measured from the finger of the same person, and training is performed in such a way as to bring these electrocardiogram waveforms close to each other. On the other hand, it is determined that the electrocardiogram waveform generated by using the pulse wave estimated from the face video is not similar to the electrocardiogram waveform generated by using the pulse wave measured from the finger of a different person, and training is performed in such a way as to separate these electrocardiogram waveforms.

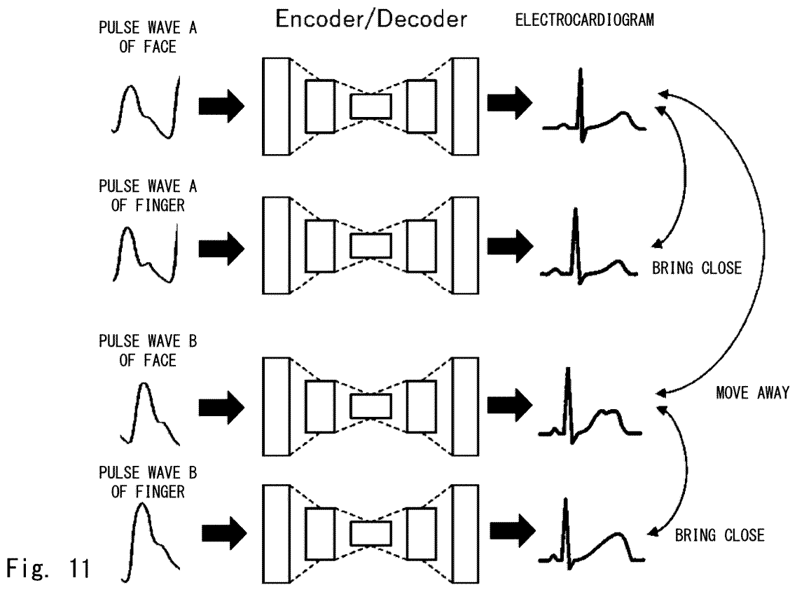

FIG. 11 is a diagram illustrating an example of training the machine learning model used by the electrocardiogram estimation unit 23.

In the second modified example, four Encoders/Decoders are used. Learning weights (parameter values of a learning target) of these four Encoders/Decoders may be shared or may not be shared.

The output from the Attention Network is input to each of the four Encoders/Decoders, and each of the four Encoders/Decoders outputs an estimated value of the electrocardiogram.

The information processing apparatus 20 applies an electrocardiogram output from each of the four Encoders/Decoders to a loss function L_{cons} represented by Formula (7), and performs training of the four Encoders/Decoders in such a way that the value of the loss function L_{cons} becomes as small as possible.

$$\begin{matrix}[ Mathematical formula 7 ] & \\ {L}_{cons} = - \frac{1}{N} {\sum }_{i = 1}^{N} log \frac{exp ( \frac{sim ( f ( {y}_{í}^{face} ) , g ( {y}_{i}^{finger} ) )}{τ} )}{{\sum }_{j = 1}^{N} exp ( \frac{sim ( f ( {y}_{i}^{face} ) , f ( {y}_{j}^{face} ) )}{τ} )} & ( 7 )\end{matrix}$$

Here, N represents the number of people in mini-batch. An electrocardiogram generated by a face video acquired from the i-th person is represented as yᵢ^{face}, and an electrocardiogram generated by a pulse wave of the finger acquired from the i-th person is represented as yᵢ^{finger}. Here, i is an integer of 1≤i≤N indicating an index for identifying a person in the mini-batch. yᵢ^{face} and yᵢ^{finger} represented in the numerator on the right side of Formula (7) indicate the electrocardiogram generated for the same person, and are an electrocardiogram of which a waveform is desired to be brought close. yᵢ^{face} and yⱼ^{face} represented in the denominator on the right side of Formula (7) represent the electrocardiograms generated for different persons, and are electrocardiograms whose waveforms are desired to be separated.

f(⋅) and g(⋅) indicate encoders configured by three convolution layers that extract feature vectors from an electrocardiogram. f(⋅) represents an encoder that extracts a feature vector from an electrocardiogram generated by a face video. g(⋅) represents an encoder that extracts a feature vector from an electrocardiogram generated by a pulse wave acquired from a finger.

sim(⋅) is a function for calculating similarity between feature vectors. The cosine similarity may be used as sim(⋅), or other similarity calculation formulas may be used. τ is a parameter for adjusting a scale of a gradient of loss. τ can be regarded as a parameter for adjusting the shape of the softmax distribution in a temperature softmax function.

By using the loss function represented by Formula (7), the information processing apparatus 20 performs training of the entire electrocardiogram estimation model in such a way that the electrocardiograms generated from the face video of the same person and the pulse wave of the finger have similar waveforms, and performs training of the entire electrocardiogram estimation model in such a way that the electrocardiograms generated from face videos of different persons have different waveforms. In particular, the information processing apparatus 20 performs not only adjustment of the parameter values of the four Encoders/Decoders but also adjustment of the parameter value of the STN and adjustment of the parameter value of the Attention Network by using the loss function represented by Formula (7).

During the operation of the information processing apparatus 20, the number of Encoders/Decoders included in the PPG2ECG model can be set to one. For example, any one of the four Encoders/Decoders illustrated in FIG. 11 may be used during the operation of the information processing apparatus 20.

In a case where the Encoder/Decoder has different parameter values during the training, the parameter value of the Encoder/Decoder selected as the Encoder/Decoder during the operation may be adopted as it is. Alternatively, the parameter values of the Encoder/Decoder used during the operation of the information processing apparatus 20 may be calculated by using the parameter values of the four Encoders/Decoders, such as taking an average of the parameter values of the four Encoders/Decoders.

Alternatively, during the operation of the information processing apparatus 20, the PPG2ECG model may include a plurality of Encoders/Decoders. For example, the PPG2ECG model may include all four Encoders/Decoders. In this case, the electrocardiogram estimation unit 23 may calculate the estimated value of the electrocardiogram by using electrocardiograms output from a plurality of Encoders/Decoders, such as taking an average of the electrocardiograms output from the plurality of Encoders/Decoders.

### Modified Example 3

The electrocardiogram estimation unit 23 may output other types of data in addition to or instead of the estimated value of the electrocardiogram. For example, by using the estimated value of the electrocardiogram, the electrocardiogram estimation unit 23 may perform at least one of estimation of the heart rate, determination as to whether there is bradycardia, determination as to whether there is tachycardia, determination as to whether there is extrasystole, and estimation of a risk of a cardiovascular disease.

For example, the electrocardiogram estimation unit 23 may estimate the heart rate by detecting an interval (RR interval) of Rpeaks of the estimated electrocardiogram. Specifically, the electrocardiogram estimation unit 23 may calculate the heart rate by the heart rate=60/RR interval.

The electrocardiogram estimation unit 23 may determine whether there are bradycardia, tachycardia, and extrasystole, or whether there are at least one or more of bradycardia, tachycardia, and extrasystole, based on the interval of Rpeaks of the estimated electrocardiogram.

For example, the electrocardiogram estimation unit 23 may detect a case where the number of Rpeaks is equal to or less than 50 per minute, as bradycardia. The electrocardiogram estimation unit 23 may detect a case where the number of Rpeaks is equal to or more than 100 per minute, as tachycardia. The electrocardiogram estimation unit 23 may detect a case where the time interval of Rpeaks suddenly jumps (a case where a change amount of the time interval of Rpeaks is larger than a predetermined threshold value), as extrasystole.

The electrocardiogram estimation unit 23 may input the estimated electrocardiogram to a trained cardiovascular disease risk estimation model, and calculate an estimated value of the risk of cardiovascular disease. The cardiovascular disease risk estimation model may be a machine learning model that is trained by using, as training data, an electrocardiogram in a case where there is a cardiovascular disease and an electrocardiogram in a case where there is no cardiovascular disease, and receives an input of the electrocardiogram and outputs a risk of the cardiovascular disease (for example, the probability that there is a cardiovascular disease).

As described above, the pulse wave estimation unit 22 estimates the pulse wave of a living body based on a video obtained by capturing the living body as an electrocardiogram estimation target by using the machine learning model trained by using a combination of the video obtained by capturing the living body and measurement data of the pulse wave of the living body as the training data.

The electrocardiogram estimation unit 23 estimates the electrocardiogram of the living body as the electrocardiogram estimation target based on the estimated pulse wave.

According to the information processing apparatus 20, the electrocardiogram can be estimated based on the video of the living body as the electrocardiogram estimation target. As a result, the information processing apparatus 20 can estimate the electrocardiogram in a non-contact manner with the living body as the estimation target of the electrocardiogram.

The information processing apparatus 20 estimating the pulse wave based on the video can be regarded as extracting a feature amount having a strong correlation with the electrocardiogram from the video. According to the information processing apparatus 20, in this respect, it is expected that the electrocardiogram can be estimated with relatively high accuracy.

Here, as a method of estimating the electrocardiogram, a method of directly estimating the electrocardiogram from a video of a living body (without passing through the estimation of a pulse wave) is also conceivable. For example, it is conceivable to perform face detection on a face video (RGB time-series signals of a face), extract face feature points, and estimate an electrocardiogram using RGB time-series signals cut out in the ROI by using the detected feature points of a contour.

However, there is no direct relationship between the face video and the electrocardiogram. For example, there is a relationship between the face video and the pulse wave, that a peripheral blood vessel that transmits a pulse wave exists in the face, but there is no such a relationship between the face video and the electrocardiogram. Therefore, in the method of directly estimating the electrocardiogram from the video of the living body, it is conceivable that the estimation accuracy of the electrocardiogram becomes relatively low.

On the other hand, the information processing apparatus 20 estimates a pulse wave from a video of a living body and estimates an electrocardiogram from the estimated pulse wave.

Regarding the estimation of the pulse wave from the video of the living body, there is a relationship between the video of the living body and the pulse wave, that there is a peripheral blood vessel that transmits the pulse wave in the vicinity of the surface of the living body to be captured, such as the face. As described above, the relationship between the video of the living body and the pulse wave can be regarded as being more direct than the relationship between the video of the living body and the electrocardiogram. In this respect, it is expected that the estimation can be performed with higher accuracy in a case where the pulse wave is estimated from the video of the living body than in a case where the electrocardiogram is estimated from the video of the living body.

Regarding the estimation of the electrocardiogram from the pulse wave, the pressure change of the blood flow in the blood vessel, which is indicated by the pulse wave, is greatly affected by contraction or relaxation of the heart. Since contraction and relaxation of the heart are controlled by the electrical signal of the heart, it can be understood that there is a strong correlation between the pulse wave and the electrocardiogram. In this respect, in a case where the electrocardiogram is estimated from the pulse wave, it is expected that the electrocardiogram can be estimated with high accuracy.

As described above, since the information processing apparatus 20 estimates the pulse wave from the video of the living body and estimates the electrocardiogram from the estimated pulse wave, it is expected that the electrocardiogram can be estimated with higher accuracy than a case where the electrocardiogram is directly estimated from the video of the living body.

The pulse wave estimation unit 22 estimates feature points of the face from the video obtained by capturing the living body as the electrocardiogram estimation target, and estimates the pulse wave of the living body as the electrocardiogram estimation target cut out based on the estimated feature point.

According to the information processing apparatus 20, it is possible to estimate the pulse wave based on a part where an influence of the pulse wave is likely to appear, such as a part under the eyes, in the video, and to estimate the electrocardiogram based on the pulse wave. According to the information processing apparatus 20, in this respect, it is expected that the electrocardiogram can be estimated with relatively high accuracy.

The electrocardiogram estimation unit 23 adjusts the phase and the amplitude of the estimated pulse wave.

According to the information processing apparatus 20, it is possible to estimate the electrocardiogram while reducing the difference in phase and amplitude for each pulse wave. According to the information processing apparatus 20, in this respect, it is expected that the electrocardiogram can be estimated with relatively high accuracy.

The electrocardiogram estimation unit 23 performs weighting to emphasize a part of the estimated pulse wave, to which attention is paid in electrocardiogram estimation, by using the attention network.

According to the information processing apparatus 20, it is expected that the electrocardiogram can be estimated with relatively high accuracy in terms of weighting the pulse wave.

The electrocardiogram estimation unit 23 estimates the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

According to the information processing apparatus 20, it is expected that the electrocardiogram can be estimated with relatively high accuracy based on the pulse wave estimated from the video obtained by capturing the living body as the electrocardiogram estimation target, in terms of using the machine learning model trained in such a way as to reduce the difference between the electrocardiogram estimated from the pulse wave estimated from the video and the measured electrocardiogram.

The electrocardiogram estimation unit 23 estimates the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between the pulse wave and a pulse wave estimated based on an electrocardiogram estimated based on the pulse wave is reduced.

According to the information processing apparatus 20, it is expected that the electrocardiogram can be estimated with relatively high accuracy in terms of estimating the electrocardiogram by using the machine learning model trained in such a way as to restore the pulse wave.

The electrocardiogram estimation unit 23 estimates the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

According to the information processing apparatus 20, by estimating the electrocardiogram by using the machine learning model trained as described above, it is expected that an electrocardiogram similar to the case of estimating the electrocardiogram based on the measurement data of the pulse wave can be estimated from the video obtained by capturing the living body. In addition, according to the information processing apparatus 20, it is expected that the electrocardiogram capturing the feature of each living body can be estimated. According to the information processing apparatus 20, in this respect, it is expected that the electrocardiogram can be estimated with relatively high accuracy.

The electrocardiogram estimation unit 23 performs, based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

According to the information processing apparatus 20, not only the estimated electrocardiogram but also an estimation result of the heart rate, a determination result of the presence or absence of bradycardia, a determination result of the presence or absence of tachycardia, a determination result of the presence or absence of extrasystole, and an estimation result of a risk of a cardiovascular disease, or a part of the above results can be presented.

### Second Example Embodiment

FIG. 12 is a diagram illustrating an example of a configuration of an information processing apparatus according to the present disclosure. In the configuration illustrated in FIG. 12, an information processing apparatus 610 includes a pulse wave estimation unit 611 and an electrocardiogram estimation unit 612.

With such a configuration, the pulse wave estimation unit 611 estimates a pulse wave of a living body based on a video obtained by capturing the living body as an electrocardiogram estimation target by using a machine learning model trained by using a combination of the video obtained by capturing the living body and measurement data of the pulse wave of the living body as training data.

The electrocardiogram estimation unit 612 estimates an electrocardiogram of the living body as the electrocardiogram estimation target based on the estimated pulse wave.

The pulse wave estimation unit 611 is an example of pulse wave estimation means. The electrocardiogram estimation unit 612 is an example of electrocardiogram estimation means.

According to the information processing apparatus 610, the electrocardiogram can be estimated based on the video of the living body as the electrocardiogram estimation target. As a result, the information processing apparatus 610 can estimate the electrocardiogram in a non-contact manner with the living body as the estimation target of the electrocardiogram.

The information processing apparatus 610 estimating the pulse wave based on the video can be regarded as extracting a feature amount having a strong correlation with the electrocardiogram from the video. According to the information processing apparatus 610, in this respect, it is expected that the electrocardiogram can be estimated with relatively high accuracy.

In particular, similarly to the above description of the information processing apparatus 20, the information processing apparatus 610 estimates the pulse wave from the video of the living body and estimates the electrocardiogram from the estimated pulse wave, and thus it is expected that the electrocardiogram can be estimated with higher accuracy than a case where the electrocardiogram is directly estimated from the video of the living body.

### Third Example Embodiment

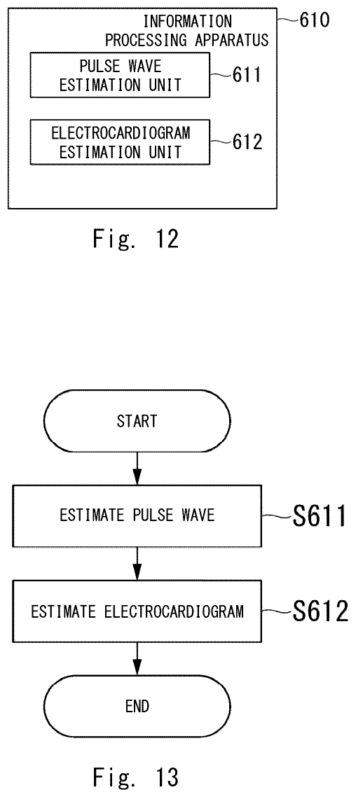

FIG. 13 is a diagram illustrating an example of a procedure of processing in an information processing method according to the present disclosure. The information processing method illustrated in FIG. 13 includes estimating a pulse wave (Step S611) and estimating an electrocardiogram (Step S612).

In the estimating of the pulse wave (Step S611), a computer estimates a pulse wave of a living body based on a video obtained by capturing the living body as an electrocardiogram estimation target by using a machine learning model trained by using a combination of the video obtained by capturing the living body and measurement data of the pulse wave of the living body as training data.

In the estimating of the electrocardiogram (Step S612), the computer estimates the electrocardiogram of the living body as the electrocardiogram estimation target based on the estimated pulse wave.

According to the information processing method illustrated in FIG. 13, the electrocardiogram can be estimated based on the video of the living body as the electrocardiogram estimation target. As a result, in the information processing method illustrated in FIG. 13, the electrocardiogram can be estimated in a non-contact manner with the living body as the estimation target of the electrocardiogram.

The estimating of the pulse wave based on the video in the information processing method illustrated in FIG. 13 can be regarded as extracting a feature amount having a strong correlation with the electrocardiogram from the video. According to the information processing method illustrated in FIG. 13, in this respect, it is expected that the electrocardiogram can be estimated with relatively high accuracy.

In particular, similarly to the above description of the information processing apparatus 20, in the information processing method illustrated in FIG. 13, the pulse wave is estimated from the video of the living body and the electrocardiogram is estimated from the estimated pulse wave, and thus it is expected that the electrocardiogram can be estimated with higher accuracy than a case where the electrocardiogram is directly estimated from the video of the living body.

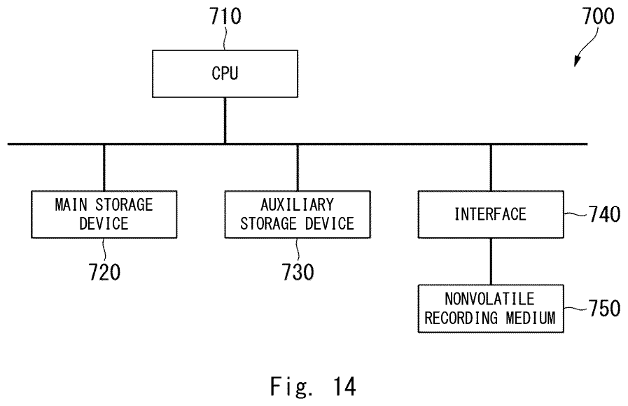

FIG. 14 is a block diagram illustrating a configuration of a computer according to the present disclosure.

In the configuration illustrated in FIG. 14, a computer 700 includes a CPU 710, a main storage device 720, an auxiliary storage device 730, an interface 740, and a nonvolatile recording medium 750.

Any one or more of the information processing apparatus 20 and the information processing apparatus 610 or a part of the apparatuses may be implemented in the computer 700. In this case, the operation of each processing unit described above is stored in the auxiliary storage device 730 in the form of a program. The CPU 710 reads the program from the auxiliary storage device 730, loads the program in the main storage device 720, and executes the above processing according to the program. The CPU 710 secures a storage area related to each of the above-described storage units in the main storage device 720 according to the program. Communication between each device and another device is executed by the interface 740 having a communication function and performing communication under the control of the CPU 710. The interface 740 has a port for the nonvolatile recording medium 750, and reads information from the nonvolatile recording medium 750 and writes information to the nonvolatile recording medium 750.

In a case where the information processing apparatus 20 is implemented in the computer 700, the operations of the video acquisition unit 21, the pulse wave estimation unit 22, the electrocardiogram estimation unit 23, and the output unit 24 are stored in the auxiliary storage device 730 in the form of a program. The CPU 710 reads the program from the auxiliary storage device 730, loads the program in the main storage device 720, and executes the above processing according to the program.

The CPU 710 secures a storage area for the information processing apparatus 20 to perform processing in the main storage device 720 according to the program. Communication between the information processing apparatus 20 and another device is executed by the operation of the interface 740 having a communication function under the control of the CPU 710. The interaction between the information processing apparatus 20 and the user is executed by the interface 740, including an input device and an output device, in which the output device presents information to the user according to the control of the CPU 710, and the input device receives a user operation.

In a case where the information processing apparatus 610 is implemented in the computer 700, the operations of the pulse wave estimation unit 611 and the electrocardiogram estimation unit 612 are stored in the auxiliary storage device 730 in the form of a program. The CPU 710 reads the program from the auxiliary storage device 730, loads the program in the main storage device 720, and executes the above processing according to the program.

The CPU 710 secures a storage area for the information processing apparatus 610 to perform processing in the main storage device 720 according to the program. Communication between the information processing apparatus 610 and another device is executed by the operation of the interface 740 having a communication function under the control of the CPU 710. The interaction between the information processing apparatus 610 and the user is executed by the interface 740, including an input device and an output device, in which the output device presents information to the user according to the control of the CPU 710, and the input device receives a user operation.

Any one or more of the above-described programs may be recorded in the nonvolatile recording medium 750. In this case, the interface 740 may read the program from the nonvolatile recording medium 750. The CPU 710 may directly execute the program read by the interface 740, or may temporarily save the program in the main storage device 720 or the auxiliary storage device 730 and execute the program.

A program for executing all or part of the processing performed by the information processing apparatus 20 and the information processing apparatus 610 may be recorded in a computer-readable recording medium, and the processing of each unit may be performed by causing a computer system to read and execute the program recorded in the recording medium. The “computer system” herein includes an Operating System (OS) and hardware such as peripheral devices.

The “computer-readable recording medium” refers to a portable medium such as a flexible disk, a magneto-optical disk, a Read Only Memory (ROM), and a Compact Disc Read Only Memory (CD-ROM), and a storage device such as a hard disk built in a computer system. The program may be for achieving some of the functions described above, and the functions described above may be achieved in combination with a program already recorded in the computer system.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Some or all of the above example embodiments may be described as the following Supplementary Notes, but are not limited to the following.

### (Supplementary Note 1)

An information processing apparatus including:
pulse wave estimation means for estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
electrocardiogram estimation means for estimating an electrocardiogram of the living body as the electrocardiogram estimation target based on the estimated pulse wave.

### (Supplementary Note 2)

The information processing apparatus according to Supplementary Note 1, in which the pulse wave estimation means estimates a feature point of a face from the video obtained by capturing the living body as the electrocardiogram estimation target, and estimates the pulse wave of the living body as the electrocardiogram estimation target based on a region cut out based on the estimated feature point.

### (Supplementary Note 3)

The information processing apparatus according to Supplementary Note 1 or 2, in which the electrocardiogram estimation means adjusts a phase and an amplitude of the estimated pulse wave.

### (Supplementary Note 4)

The information processing apparatus according to any one of Supplementary Notes 1 to 3, in which the electrocardiogram estimation means performs weighting to emphasize a part of the estimated pulse wave, to which attention is paid in electrocardiogram estimation, by using an attention network.

### (Supplementary Note 5)

The information processing apparatus according to any one of Supplementary Notes 1 to 4, in which the electrocardiogram estimation means estimates the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

### (Supplementary Note 6)

The information processing apparatus according to any one of Supplementary Notes 1 to 5, in which the electrocardiogram estimation means estimates the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

### (Supplementary Note 7)

The information processing apparatus according to any one of Supplementary Notes 1 to 6, in which the electrocardiogram estimation means estimates the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

### (Supplementary Note 8)

The information processing apparatus according to any one of Supplementary Notes 1 to 7, in which the electrocardiogram estimation means performs, based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

### (Supplementary Note 9)

An information processing method including:
by a computer,
estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
estimating an electrocardiogram of the living body as the electrocardiogram estimation target based on the estimated pulse wave.

### (Supplementary Note 10)

The information processing method according to Supplementary Note 9, in which the estimating of the pulse wave includes, by the computer, estimating a feature point of a face from the video obtained by capturing the living body as the electrocardiogram estimation target, and estimating the pulse wave of the living body as the electrocardiogram estimation target based on a region cut out based on the estimated feature point.

### (Supplementary Note 11)

The information processing method according to Supplementary Note 9 or 10, in which the estimating of the electrocardiogram includes, by the computer, adjusting a phase and an amplitude of the estimated pulse wave.

### (Supplementary Note 12)

The information processing method according to any one of Supplementary Notes 9 to 11, in which the estimating of the electrocardiogram includes, by the computer, performing weighting to emphasize a part of the estimated pulse wave, to which attention is paid in electrocardiogram estimation, by using an attention network.

### (Supplementary Note 13)

The information processing method according to any one of Supplementary Notes 9 to 12, in which the estimating of the electrocardiogram includes, by the computer, estimating the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

### (Supplementary Note 14)

The information processing method according to any one of Supplementary Notes 9 to 13, in which the estimating of the electrocardiogram includes, by the computer, estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

### (Supplementary Note 15)

The information processing method according to any one of Supplementary Notes 9 to 14, in which the estimating of the electrocardiogram includes, by the computer, estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

### (Supplementary Note 16)

The information processing method according to any one of Supplementary Notes 9 to 15, in which the estimating of the electrocardiogram includes, by the computer, performing, based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

### (Supplementary Note 17)

A program for causing a computer to execute:
estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
estimating an electrocardiogram of the living body as the electrocardiogram estimation target based on the estimated pulse wave.

### (Supplementary Note 18)

The program according to Supplementary Note 17, in which in the estimating of the pulse wave, the computer is caused to execute estimating a feature point of a face from the video obtained by capturing the living body as the electrocardiogram estimation target, and estimating the pulse wave of the living body as the electrocardiogram estimation target based on a region cut out based on the estimated feature point.

### (Supplementary Note 19)

The program according to Supplementary Note 17 or 18, in which the estimating of the electrocardiogram includes, by the computer, adjusting a phase and an amplitude of the estimated pulse wave.

### (Supplementary Note 20)

The program according to any one of Supplementary Notes 17 to 19, in which in the estimating of the electrocardiogram, the computer is caused to execute performing weighting to emphasize a part of the estimated pulse wave, to which attention is paid in electrocardiogram estimation, by using an attention network.

### (Supplementary Note 21)

The program according to any one of Supplementary Notes 17 to 20, in which in the estimating of the electrocardiogram, the computer is caused to execute estimating the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

### (Supplementary Note 22)

The program according to any one of Supplementary Notes 17 to 21, in which in the estimating of the electrocardiogram, the computer is caused to execute estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

### (Supplementary Note 23)

The program according to any one of Supplementary Notes 17 to 22, in which in the estimating of the electrocardiogram, the computer is caused to execute estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

### (Supplementary Note 24)

The program according to any one of Supplementary Notes 17 to 23, in which in the estimating of the electrocardiogram, the computer is caused to execute performing, based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

1. An information processing apparatus comprising:
at least one memory storing instructions; and
at least one processor configured to execute the instructions to:
estimate a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
estimate an electrocardiogram of the living body as the electrocardiogram estimation target based on the estimated pulse wave,
estimate a feature point of a face from the video obtained by capturing the living body as the electrocardiogram estimation target, and estimate the pulse wave of the living body as the electrocardiogram estimation target based on a region cut out based on the estimated feature point, and
perform weighting to emphasize a part of the estimated pulse wave, to which attention is paid in electrocardiogram.

2. The information processing apparatus according to claim 1, wherein the at least one processor is configured to execute the instructions to adjust a phase and an amplitude of the estimated pulse wave.

3. The information processing apparatus according to claim 1, wherein the at least one processor is configured to execute the instructions to estimate the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

4. The information processing apparatus according to claim 1, wherein the at least one processor is configured to execute the instructions to estimate the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

5. The information processing apparatus according to claim 1, wherein at least one processor configured to execute the instructions to estimate the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

6. The information processing apparatus according to claim 1, wherein at least one processor configured to execute the instructions to perform, based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

7. An information processing method comprising:
by a computer,
estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
estimating an electrocardiogram of the living body as the electrocardiogram estimation target based on the estimated pulse wave,
estimating a feature point of a face from the video obtained by capturing the living body as the electrocardiogram estimation target, and estimating the pulse wave of the living body as the electrocardiogram estimation target based on a region cut out based on the estimated feature point, and wherein
performing weighting to emphasize a part of the estimated pulse wave, to which attention is paid in electrocardiogram.

8. The information processing method according to claim 7, further comprising: by the computer,
adjusting phase and an amplitude of the estimated pulse wave.

9. The information processing method according to claim 7, further comprising: by the computer,
estimating the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

10. The information processing method according to claim 7, further comprising: by the computer,
estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

11. The information processing method according to claim 7, further comprising: by the computer,
estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

12. The information processing method according to claim 7, further comprising: by the computer,
performing based on the estimated electrocardiogram, at least one of estimation of a heart rate, determination as to whether there is bradycardia, determination as to whether there is tachycardia, determination as to whether there is extrasystole, and estimation of a risk of a cardiovascular disease.

13. A non-transitory computer readable medium storing a program for causing a computer to execute:
estimating a pulse wave of a living body as an electrocardiogram estimation target based on a video obtained by capturing the living body as the electrocardiogram estimation target, by using a machine learning model trained using, as training data, a combination of a video obtained by capturing a living body and measurement data of a pulse wave of the living body; and
estimating an electrocardiogram of the living body as the electrocardiogram estimation target based on the estimated pulse wave,
estimating a feature point of a face from the video obtained by capturing the living body as the electrocardiogram estimation target, and estimating the pulse wave of the living body as the electrocardiogram estimation target based on a region cut out based on the estimated feature point, and wherein
performing weighting to emphasize a part of the estimated pulse wave, to which attention is paid in electrocardiogram.

14. The non-transitory computer readable medium according to claim 13, storing the program for causing the computer to further execute:
adjusting phase and an amplitude of the estimated pulse wave.

15. The non-transitory computer readable medium according to claim 13, storing the program for causing the computer to further execute:
estimating the electrocardiogram of the living body as the electrocardiogram estimation target based on the pulse wave estimated from the video obtained by capturing the living body, by using a machine learning model trained in such a way that a difference between an electrocardiogram estimated based on a pulse wave estimated from a video obtained by capturing a living body and an electrocardiogram measured from the living body is reduced.

16. The non-transitory computer readable medium according to claim 13, storing the program for causing the computer to further execute:
estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between a pulse wave estimated from a video obtained by capturing a living body and a pulse wave estimated again from an electrocardiogram estimated based on the pulse wave estimated from the video is reduced.

17. The non-transitory computer readable medium according to claim 13, storing the program for causing the computer to further execute:
estimating the electrocardiogram of the living body based on the pulse wave estimated based on the video obtained by capturing the living body as the electrocardiogram estimation target, by using a machine learning model for estimating an electrocardiogram based on a pulse wave, the machine learning model being trained in such a way that a difference between an electrocardiogram estimated based on a pulse wave and an electrocardiogram estimated based on a pulse wave estimated based on a video is reduced in a case where a living body from which the pulse wave has been measured is the same as a living body of which the video has been captured, and being trained in such a way that the difference between the electrocardiogram estimated based on the pulse wave and the electrocardiogram estimated based on the pulse wave estimated based on the video is increased in a case where the living body from which the pulse wave has been measured is different from the living body of which the video has been captured.

18. The non-transitory computer readable medium according to claim 13, storing the program for causing the computer to further execute:
performing based on the estimated electrocardiogram, at least one of estimation of a heart rate, determination as to whether there is bradycardia, determination as to whether there is tachycardia, determination as to whether there is extrasystole, and estimation of a risk of a cardiovascular disease.
